(19) United States
(12) Patent Application Publication
(10) Pub. No.: US 2023/0084335 A1
(43) Pub. Date: Mar. 16, 2023

(54) ANALYSIS METHOD FOR PREDICTION OF RESPONSE TO PREOPERATIVE CHEMORADIOTHERAPY IN RECTAL CANCER PATIENT

(71) Applicants: THE ASAN FOUNDATION, Seoul (KR); UNIVERSITY OF ULSAN FOUNDATION FOR INDUSTRY COOPERATION, Ulsan (KR); CBSBIOSCIENCE CO., LTD, Daejeon (KR)

(72) Inventors: Chang-Sik YU, Seoul (KR); In-Ja PARK, Seoul (KR); Seung-Mo HONG, Seoul (KR); Sung-Min AHN, Seoul (KR); Adnan Ahmad ANSARI, Incheon (KR); Bilal MUSTAFA, Incheon (KR); Jin-Young PARK, Seoul (KR)

(73) Assignees: THE ASAN FOUNDATION, Seoul (KR); UNIVERSITY OF ULSAN FOUNDATION FOR INDUSTRY COOPERATION, Ulsan (KR); CBSBIOSCIENCE CO., LTD, Daejeon (KR)

(21) Appl. No.: 17/904,678

(22) PCT Filed: Feb. 8, 2021
(86) PCT No.: PCT/KR2021/001605

§ 371 Date: Sep. 1, 2022

(30) Foreign Application Priority Data
Feb. 21, 2020 (KR) 10-2020-0021586

(51) Int. Cl. C12Q 1/6886

(52) U.S. Cl. CPC C12Q 1/6886; C12Q 2600/158

(57) ABSTRACT

Provided are an analytical method of providing an information for diagnosis of a patient who exhibits a response to preoperative chemoradiotherapy in a rectal cancer patient, wherein the expression levels of nine genes, i.e., FGFR3, GNA11, H3F3A, IL12A, IL1R1, IL2RB, NKD1, SGK2, and SPRY2 genes, is analyzed in combination, in carcinoma tissues of a rectal cancer patient, the response (susceptibility) to preoperative chemoradiotherapy can be predicted with high accuracy, and therefore the combination of said genes can be usefully applied as a biomarker for selecting a rectal cancer patient who exhibits a response to preoperative chemoradiotherapy.

### TECHNICAL FIELD

The present invention relates to an analytical method for predicting the response to preoperative chemoradiotherapy in rectal cancer patients. More specifically, the present invention relates to an analytical method of providing an information for diagnosis of a patient who exhibits a response to preoperative chemoradiotherapy in a rectal cancer patient.

### BACKGROUND ART

Locally advanced rectal cancer (LARC) is described as an invasive rectal tumor is with unresectable margins involving the mesorectal fascia and clinically suspicious lymph nodes (lateral pelvic lymph nodes) (de Wilt J H, et al., Management of locally advanced primary and recurrent rectal cancer. Clin Colon Rectal Surg 2007; 20: 255-63; Glynne-Jones R, et al., Locally advanced rectal cancer: what is the evidence for induction chemoradiation? Oncologist 2007; 12: 1309-18). Preoperative chemoradiotherapy (PCRT) followed by surgical resection is the standard multimodal treatment for LARC (Kapiteijn E, et al., Preoperative radiotherapy combined with total mesorectal excision for resectable rectal cancer. N Engl J Med 2001; 345: 638-46; Rodel C., et al., Radiotherapy: Preoperative chemoradiotherapy for rectal cancer. Nat Rev Clin Oncol 2010; 7: 129-30). PCRT decreases the risk of local recurrence and increases the possibility of sphincter preservation. Patients who respond favorably to PCRT show good oncologic outcomes (Fokas E, et al., Tumor regression grading after preoperative chemoradiotherapy for locally advanced rectal carcinoma revisited: updated results of the CAO/ARO/AlO-94 trial. J Clin Oncol 2014; 32: 1554-62; Park I J, et al., Neoadjuvant treatment response as an early response indicator for patients with rectal cancer. J Clin Oncol 2012; 30: 1770-6).

In patients who show a complete or near-complete response to PCRT, this treatment enables rectal-sparing surgical treatment of the tumor. Although PCRT followed by surgical resection is the standard treatment for LARC, approximately two-thirds of patients show partial or no-response to PCRT; in these patients, PCRT does not improve the clinical outcome (Lee Y C et al., Prognostic significance of partial tumor regression after preoperative chemoradiotherapy for rectal cancer: a meta-analysis. Dis Colon Rectum 2013; 56: 1093-101). Moreover, PCRT is associated with two adverse effects in non-responders: 1) radiation therapy is associated with long-term complications that affect the quality of life of patients; and 2) delayed surgical resection due to PCRT may lead to local and distant tumor spread (Conde-Muino R, et al. Predictive Biomarkers to Chemoradiation in Locally Advanced Rectal Cancer. Biomed Res Int 2015; 2015: 921435; Millino C, et al. Gene and MicroRNA Expression Are Predictive of Tumor Response in Rectal Adenocarcinoma Patients Treated With Preoperative Chemoradiotherapy. J Cell Physiol 2017; 232: 426-435). Therefore, is various efforts have been made to develop biomarkers for predicting the response to PCRT in LARC patients, which would enable the selection of responders who would benefit from PCRT.

Several studies demonstrated the potential of genetic biomarkers to accurately predict the response to and outcome of PCRT (Watanabe T, et al. Prediction of sensitivity of rectal cancer cells in response to preoperative radiotherapy by DNA microarray analysis of gene expression profiles. Cancer Res 2006; 66:3370-4; Agostini M, et al. An integrative approach for the identification of prognostic and predictive biomarkers in rectal cancer. Oncotarget 2015; 6:32561-74; Gim J, et al. Predicting multi-class responses to preoperative chemoradiotherapy in rectal cancer patients. Radiat Oncol 2016; 11: 50). Ghadimi et al. identified 54 differentially expressed genes (DEGs) between responders and non-responders, and expression profiling could predict tumor behavior in 83% of patients with LARC (p=0.02) (Ghadimi B M, et al. Effectiveness of gene expression profiling for response prediction of rectal adenocarcinomas to preoperative chemoradiotherapy. J Clin Oncol 2005; 23: 1826-38). Gantt et al. used 33 rectal cancer biopsy samples and identified two gene expression profiles that differentiated non-responders from responders (Gantt G A, et al. Gene expression profile is associated with chemoradiation resistance in rectal cancer. Colorectal Dis 2014; 16: 57-66). Guo et al. recently identified a 27 gene signature in LARC patients capable of predicting the response to PCRT based on relative expression ordering (REO) patterns (Guo Y, et al. A qualitative signature for predicting pathological response to neoadjuvant chemoradiation in locally advanced rectal cancers. Radiother Oncol 2018; 129: 149-153). And, Chauvin et al. reported the potential of proteomic profiling for predicting the response to PCRT in LARC patients (Chauvin A, et al. The response to neoadjuvant chemoradiotherapy with 5-fluorouracil in locally advanced rectal cancer patients: a predictive proteomic signature. Clin. Proteomics 2018; 15: 16; Repetto O, et al. Identification of protein clusters predictive of tumor response in rectal cancer patients receiving neoadjuvant chemo-radiotherapy. Oncotarget 2017; 8:28328-28341). Despite identification of gene expression profiles involved in LARC, biomarkers for clinical use have not been identified to date.

One of the factors limiting the development of PCRT biomarkers is the use of formalin-fixed paraffin-embedded (FFPE) biopsy samples collected before PCRT and surgical excision. However, fresh-frozen samples cannot be used because of factors such as tumor cellularity, necrosis, and immune infiltrates limit downstream expression analyses.

### DISCLOSURE

### Technical Problem

The present inventors have performed various studies to develop clinically applicable biomarkers (also referred to as “gene signature”) capable of predicting the response to PCRT. Especially, the present inventors used FFPE tissue samples for RNA extraction and performed gene expression analysis using FDA-approved hardware and kits. As a result, it has been found that, when analyzed in combination of the expression levels of specific genes, i.e., FGFR3, GNA11, H3F3A, IL12A, IL1R1, IL2RB, NKD1, SGK2, and SPRY2 genes, in carcinoma tissues of a rectal cancer patient, the response (also referred to as “susceptibility”) to preoperative chemoradiotherapy can be predicted with high accuracy.

Therefore, it is an object of the present invention to provide an analytical method for predicting a response to preoperative chemoradiotherapy in rectal cancer patients, the method of which comprises using FGFR3, GNA11, H3F3A, IL12A, IL1R1, IL2RB, NKD1, SGK2, and SPRY2 genes as biomarkers.

### Technical Solution

In accordance with an aspect of the present invention, there is provided an analytical method of providing an information for diagnosis of a patient who exhibits a response to preoperative chemoradiotherapy in a rectal cancer patient, the method of which comprises measuring expression levels of FGFR3, GNA11, H3F3A, IL12A, IL1R1, IL2RB, NKD1, SGK2, and SPRY2 genes, in carcinoma tissue samples which are externally discharged from the rectal cancer patient, respectively.

In the analytical method of the present invention, the rectal cancer patient may be a locally advanced rectal cancer patient.

And, in the analytical method of the present invention, the carcinoma tissue samples which are externally discharged from the rectal cancer patient may be formalin-fixed paraffin-embedded carcinoma tissue-derived biopsy samples.

### Advantageous Effects

It has been found by the present invention that, when analyzed in combination of the expression levels of specific genes, i.e., FGFR3, GNA11, H3F3A, IL12A, IL1R1, IL2RB, NKD1, SGK2, and SPRY2 genes, in carcinoma tissues of a rectal cancer patient, the response (susceptibility) to preoperative chemoradiotherapy can be predicted with high accuracy. Therefore, the combination of said genes can be usefully applied as a biomarker for selecting a rectal cancer patient who exhibits a response to preoperative chemoradiotherapy.

### DESCRIPTION OF DRAWINGS

FIG. 1 shows a flowchart of gene signature development.

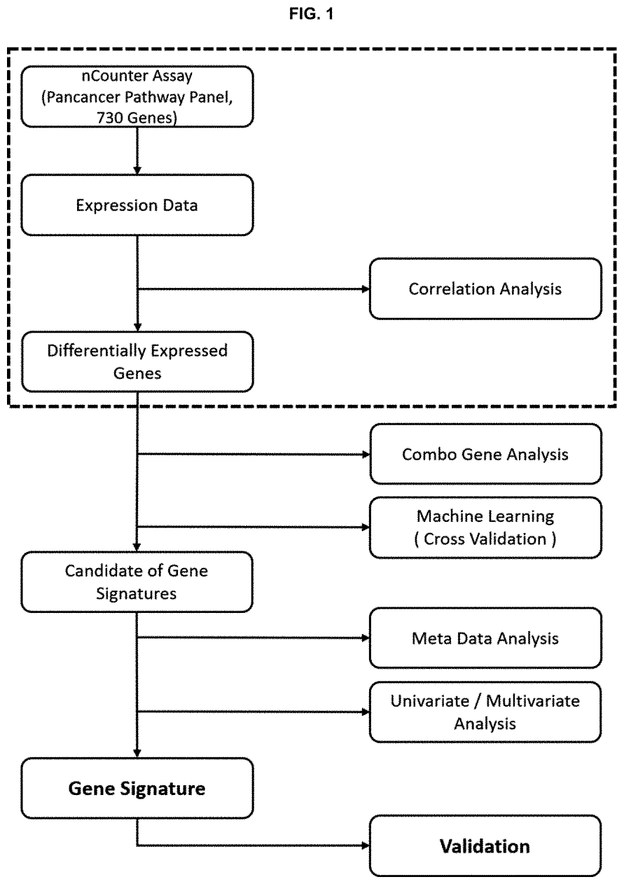

FIG. 2 shows a flowchart of meta-data analysis.

FIG. 3 shows the metadata analysis-based PCRT responder gene signature related pathway: PI3K-Akt signaling pathway.

FIG. 4 shows the PI3K-Akt signaling pathway three-dimensional genomic expression map for PCRT responders based on metadata analysis.

FIG. 5 shows an example showing uncheck of the Background Subtraction in nSolver Analysis Software v 3.0 (Nanostring Technologies) provided to nCounter (Nanostring Technologies, Seattle, Wash.).

FIG. 6 shows an example of a Normalization Parameter setting.

### BEST MODE FOR CARRYING OUT THE INVENTION

PCRT, followed by surgical resection, is the standard treatment for LARC. However, approximately two thirds of the patients show partial or no-response to PCRT, leaving PCRT-associated side effects without favorable clinical outcomes. The present inventors evaluated pathological response of the tumors in a total of 156 LARC patients (training cohort n=60; validation cohort n=96) who underwent surgical resection post PCRT; and classified the categories into responders (patients with complete or near-complete regression; n=72) and non-responders (all other patients; n=84). The RNA from the FFPE samples was subjected to gene expression analysis with nCounter (Nanostring Technologies, Seattle, Wash.). By using univariate and multivariate logistic regression, we identified a 9-gene signature that differentiated between responders and non-responders (i.e., FGFR3, GNA11, H3F3A, IL12A, IL1R1, IL2RB, NKD1, SGK2, and SPRY2 genes). The expression of 9-gene signature distinctively differentiated responders (n=27) from non-responders (n=33) in training cohort of 60 LARC samples (accuracy=86.9%, specificity=84.8%, sensitivity=81.5%). The results were validated in an independent cohort of 96 LARC patients and successfully differentiated PCRT responders from non-responders (accuracy=81.0%, specificity=79.4%, sensitivity=82.3%). The signature was independent of all pathological and clinical features. Therefore, the 9-gene signature can be used as biomarkers capable of predicting the response to PCRT in LARC patients. This gene signature is readily applicable to the clinical setting using FFPE samples and FDA-approved hardware and reagents. Tailored treatment approaches in good and poor responders to PCRT may improve the oncologic outcomes of patients with LARC.

Accordingly, the present invention provides an analytical method of providing an information for diagnosis of a patient who exhibits a response to preoperative chemoradiotherapy in a rectal cancer patient, the method of which comprises measuring expression levels of FGFR3, GNA11, H3F3A, IL12A, IL1R1, IL2RB, NKD1, SGK2, and SPRY2 genes, in carcinoma tissue samples which are externally discharged from the rectal cancer patient, respectively.

In the analytical method of the present invention, the rectal cancer patient may be preferably a locally advanced rectal cancer patient. And, in the analytical method of the present invention, the carcinoma tissue samples which are externally discharged from the rectal cancer patient may be formalin-fixed paraffin-embedded carcinoma is tissue-derived biopsy samples.

In the analytical method of the present invention, the nine genes used as biomarkers, i.e., FGFR3, GNA11, H3F3A, IL12A, IL1R1, IL2RB, NKD1, SGK2, and SPRY2 genes, are known in the art and the sequences thereof are known in GenBank and the like. For example, the NCBl Accession Numbers of the FGFR3 (Fibroblast Growth Factor Receptor 3) protein are NP_000133, NP_001156685, NP_075254, NP_001341738, NP_001341739 and the like; and the NCBl Accession Numbers of the genes encoding the same (mRNAs) are NM_000142, NM_001163213, NM_022965, NM_001354809, NM_001354810 and the like. The NCBI Accession Number of the GNA11 (Guanine nucleotide-binding protein subunit alpha-11) protein is NP_002058; and the NCBI Accession Number of the gene encoding the same (mRNA) is NM_002067. The NCBI Accession Number of the H3F3A (Histone H3.3) protein is NP_005315; and the NCBI Accession Number of the gene encoding the same (mRNA) is NM_002107. The NCBI Accession Numbers of the IL12A (Interleukin-12 subunit alpha) protein are NP_000873, NP_001341511, NP_001341512 and the like; and the NCBI Accession Numbers of the genes encoding the same (mRNAs) are NM_000882, NM_001354582, NM_001354583 and the like. The NCBI Accession Numbers of the IL1R1 (Interleukin 1 receptor, type I) protein are NP_000868, NP_001275635, NP_001307907, NP_001307909, NP_001307910 and the like; and the NCBI Accession Numbers of the genes encoding the same (mRNAs) are NM_000877, NM_001288706, NM_001320978, NM_001320980, NM_001320981 and the like. The NCBI Accession Numbers of the IL2RB (Interleukin-2 receptor subunit beta) protein are NP_000869, NP_001333151, NP_001333152 and the like; and the NCBI Accession Numbers of the genes encoding the same (mRNAs) are NM_000878, NM_001346222, NM_001346223 and the like. The NCBI Accession Number of the NKD1 (Naked cuticle 1) protein is NP_149110.1; and the NCBI Accession Number of the gene encoding the same (mRNA) is NM_033119.5. The NCBI Accession Numbers of the SGK2 (Serine/threonine-protein kinase Sgk2) protein are NP_001186193, NP_057360, NP_733794 and the like; and the NCBI Accession Numbers of the genes encoding the same (mRNAs) are NM_170693, NM_001199264, NM_016276 and the like. The NCBI Accession Numbers of the SPRY2 (Sprouty homolog 2) protein are NP_001305465, NP_001305466, is NP_001305467, NP_005833 and the like; and the NCBI Accession Numbers of the genes encoding the same (mRNAs) are NM_005842, NM_001318536, NM_001318537, NM_001318538 and the like.

In an embodiment of the analytical method according to the present invention, after measuring the expression levels of FGFR3, GNA11, H3F3A, IL12A, IL1R1, IL2RB, NKD1, SGK2, and SPRY2 genes in carcinoma tissue samples externally discharged from the rectal cancer patient; when the TBPS (Treatment Benefit Prediction Score) value calculated according to the following equation is greater than −7.269813, the patient can be classified to a patient who exhibits a response to PCRT (i.e., a patient who exhibits susceptibility to PCRT) and when the TBPS value is −7.269813 or less, the patient can be classified to a patient who does not exhibit a response to PCRT (i.e., a patient who does not exhibit susceptibility to PCRT).

TBPS=(−0.006697)*G_{FGFR3}+(−0.001805)*G_{GNA11}+(−0.000373)*G_{H3F3A}+(0.063996)*G_{IL12A}+(0.015269)*G_{IL1R1}+(0.017445)*G_{IL2RB}+(−0.003099)*G_{NKD1}+(−0.004739)*G_{SGK2}+(−0.002763)*G_{SPRY2}

In the above equation, G_{FGFR3}, G_{GNA11}, G_{H3F3A}, G_{IL12A}, G_{IL1R1}, G_{IL2RB}, G_{NKD1}, G_{SGK2}, and G_{SPRY2} represent the gene expression levels of FGFR3, GNA11, H3F3A, IL12A, IL1R1, IL2RB, NKD1, SGK2, and SPRY2 genes, respectively. That is, the expression level of each gene represents a normalized expression level obtained by using nCounter (Nanostring Technologies, Seattle, Wash.), a gene expression measuring device. The normalization is performed according to the manufacturer's protocol, using nSolver Analysis Software v 3.0 (Nanostring Technologies), which is provided to nCounter (Nanostring Technologies, Seattle, Wash.).

The present invention will be described in further detail with reference to the following examples. These examples are for illustrative purposes only and are not intended to limit the scope of the present invention.

1. Test Methods

(1) RNA Extraction

Total RNA was extracted from FFPE tissues (n=156) using the RNeasy FFPE kit (Qiagen, Hilden, Germany) with Deparaffinization Solution (Qiagen) and DNase I treatment (Qiagen). Written consent was obtained from patients before enrollment, and the study's retrospective protocol was approved by the Institutional Review Board of Asan Medical Center (2017-0333).

(2) Patients and Response Assessment

The study included 156 randomly selected rectal cancer patients (n=156) divided into a training cohort (n=60) and a validation cohort (n=96). All patients underwent surgical resection after PCRT between January 2014 and December 2017 at Asan Medical Center, Seoul, Korea. Patients were excluded if they did not undergo surgical treatment, had no available pretreatment biopsy specimens, or could not undergo post-treatment pathological response assessment.

Preoperative chemoradiotherapy was delivered at a dose of 45-50.4 Gy in 25 or 28 fractions. Chemotherapy consisted of two cycles of intravenous 5-fluorouracil (375 mg/m² daily) plus leucovorin (20 mg/m² daily) in a bolus administered over 3 days during the 1st and 5th weeks of RT, and oral capecitabine (1,650 mg/m² daily) administered twice per day during radiotherapy. Surgical resection was performed 6-8 weeks after completion of PCRT. The surgical resection included local excision and radical resection performed according to the principles of total mesorectal excision (TME) (Heald R J, et al., The mesorectum in rectal cancer surgery—the clue to pelvic recurrence? Br J Surg 1982; 69: 613-6).

The five-tier classification for tumor regression grading (TRG) was used to evaluate the pathological responses of the primary tumor to PCRT (Mandard A M, et al. Pathologic assessment of tumor regression after preoperative chemoradiotherapy of esophageal carcinoma. Clinicopathologic correlations. Cancer 1994; 73: 2680-6). The TRG categories, which were selected according to the residual tumor and fibrosis, were as follows: 1) complete regression (no residual tumor cells and only a fibrotic mass), 2) near-complete regression (difficult to microscopically find residual tumor cells in the fibrotic tissue), 3) moderate regression (easily identifiable dominant irradiation-related changes with residual tumor), 4) minimal regression (a dominant tumor mass with obvious irradiation-related changes), and 5) no regression (no evidence of irradiation-related fibrosis, necrosis, or vascular changes). Study subjects were classified into two broad classes: responders (patients with complete or near-complete regression; n=72) and non-responders (all other patients; n=84).

(3) Gene Expression Assay

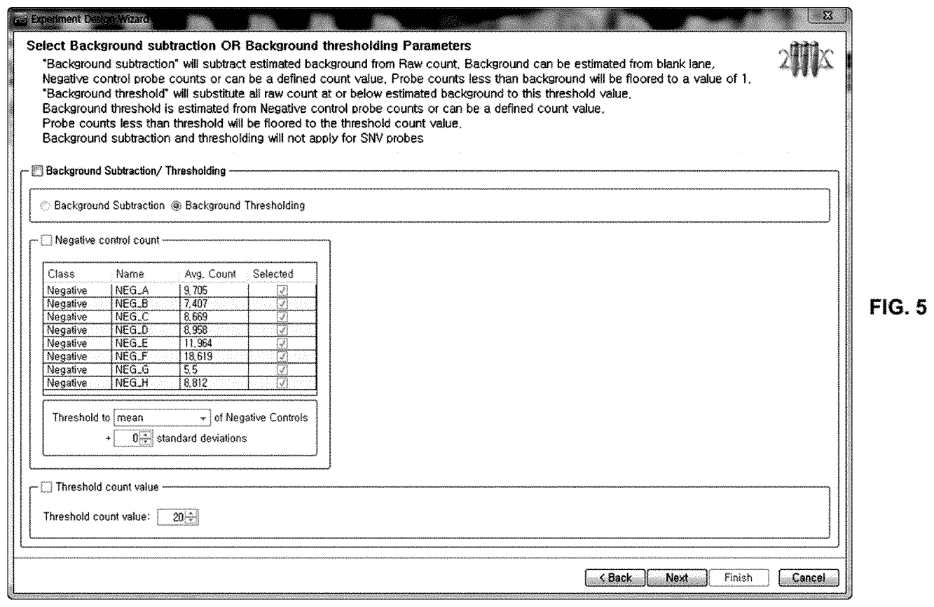

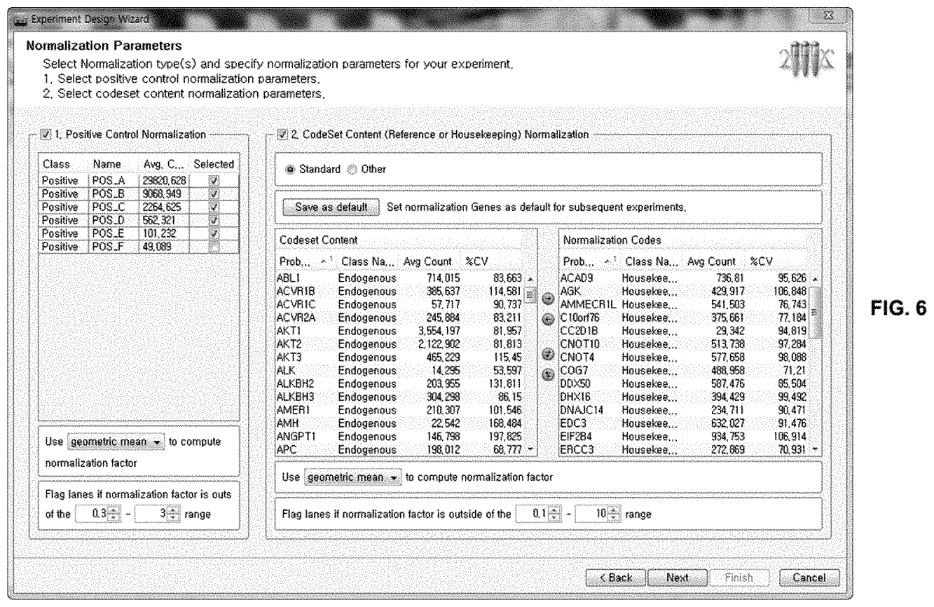

Gene expression analysis for total 770 genes (including 730 endogenous genes and 40 housekeeping genes) was performed using nCounter (Nanostring Technologies, Seattle, Wash.). The reaction in each panel contained 15 μL of Aliquot, which included 200 ng of total RNA, reporter probes and capture probes. Normalization of the gene expression raw data was performed according to the manufacturer's protocol using nSolver Analysis Software v 3.0 (Nanostring Technologies) provided to nCounter (Nanostring Technologies, Seattle, Wash.). Specifically, after selecting the sample to be analyzed in the tested samples, the Background Subtraction was unchecked. FIG. 5 shows an example showing uncheck of the Background Subtraction in nSolver Analysis Software v 3.0. In the normalization parameters, the POS_F was unchecked, the geometric mean was selected, and the Range was set to 0.3-3 in Positive Control Normalization. The CodeSet Content (Reference or Housekeeping) normalization was set to Standard. The endogenous genes were selected as the Codeset Content and the housekeeping genes were selected as the Normalization Codes. The geometric mean was selected and the Range was set to 0.1-10. FIG. 6 shows an example of a Normalization Parameter setting.

(4) Statistical Analysis

The clinicopathological variables of the training and validation cohorts were evaluated using the χ²-test and Fisher's exact test, and a p<0.05 was considered statistically significant.

(5) Statistical Combination Gene Analysis

All statistical analyses in this study were performed using the open source statistical programming environment R language (version 3.4.3). In the training cohort, the Student's t-test was used to classify DEGs as over- or under-expressed (p<0.05 and |fold-change|>1.5) to compare PCRT treatment responders with non-responders. DEGs were further shortlisted using univariate logistic regression (p<0.05). The number of shortlisted DEGs analyzed in combination and the total number of gene combinations was calculated using the following equation:

$${{\sum }_{k = 1}}^{n} \frac{n !}{K ! ( n - k ) !}$$

In the above equation, n is the total number of shortlisted DEGs and k is the number of genes included in the combinations.

Multivariate logistic regression analysis was used to measure the association between gene signatures and clinicopathological features (p<0.05; Table 5).

The candidate gene signatures (p<0.05; AUC>0.08; sensitivity>75%; and specificity>75%) were ranked by k-fold cross validation to identify the optimal gene combination. The training cohorts were separated into two folds (training sets and test sets) and the results were validated by applying the reference value in the training set to the test set. The patient groups of the training set and the test set were randomly divided and tested 300 times. The accuracy was calculated based on a p<0.05 on the test set.

(6) Meta-Analysis

A meta-analysis of the training cohort (n=60) was performed to identify signal transduction pathways activated in PCRT responders. The meta-analysis was performed using CBS Probe PINGS™ (Protein Interaction Network Generation System, Korean Patent No. 10-0957386). The program uses five modules to identify interacting genes and gene interaction information for gene combinations, such as interaction distance and interaction frequency: Protein-Protein interactions module (PPI module), Path-Finder module, Path-Linker module, Path-maker module and Path-Lister module. The identified genes were mapped to the signal transduction pathways obtained from the Kyoto Encyclopedia of Genes and Genomes (KEGG) database (Kanehisa M, et al. The KEGG databases at GenomeNet. Nucleic Acids Res. 2002; 30: 42-6). The top ten signal transduction pathways were selected according to the number of interactions and interacting genes.

A meta-analysis of the training cohort was performed to identify the most significant transduction pathways in PCRT responders by calculating the number of combination genes related to these signal transduction pathways.

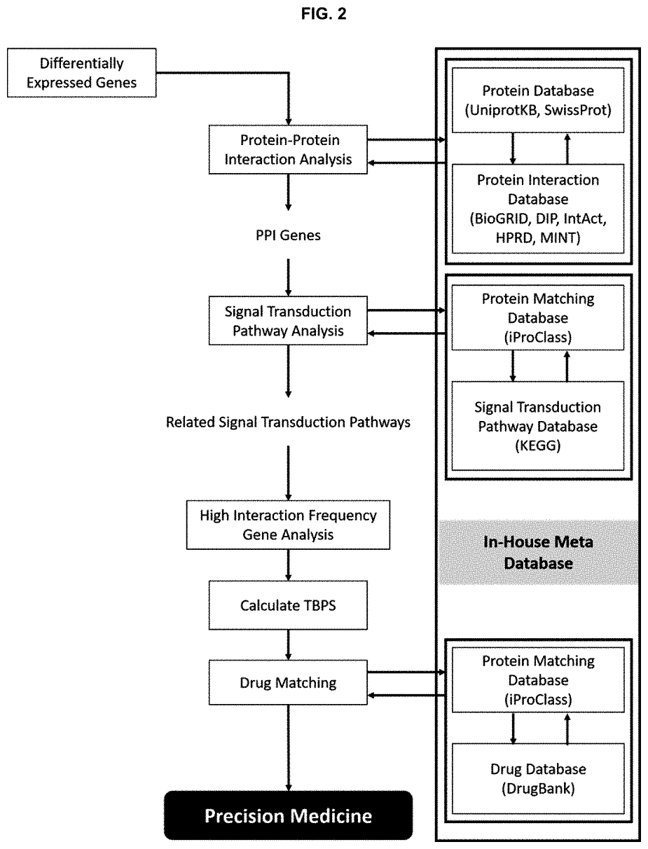

For each signal transduction pathway, we computed the gene interaction is frequency of interacting genes with signature genes. A gene interaction frequency of 100% indicated the highest probability of gene interaction within each signal transduction pathway. A threshold of 75% was selected as a high interaction frequency (FIG. 2). The top ten signal transduction pathways having high interaction frequency genes were identified in each patient in the training cohort, and gene signatures were matched to the related signal transduction pathways.

2. Test Results

(1) Clinicopathological Features

Table 1 summarizes the clinicopathological features of the study cohorts. There were no statistically significant differences between the training cohort and the validation cohort.

TABLE 1
Clinicopathological features in the training and validation cohorts

| | | Training cohort | Validation cohort | |
|---|---|---|---|---|
| | | (n = 60) | (n = 96) | p-value |
| Gender | Male | 27 (45.0%) | 53 (55.2%) | 0.282 |
| | Female | 33 (55.0%) | 43 (44.8%) | |
| Differentia- | Well | 9 (15%) | 18 (18.9%) | 0.596 |
| tion Grade | Moderately | 51 (85.0%) | 76 (79.1%) | |
| | Poorly | 0 (0.0%) | 2 (2.0%) | |
| Clinical | T1 | 0 (0.0%) | 0 (0.0%) | 0.810 |
| T-stage | T2 | 4 (6.7%) | 9 (9.4%) | |
| | T3 | 53 (88.3%) | 78 (81.3%) | |
| | T4 | 3 (5.0%) | 4 (9.0%) | |
| Clinical | N0 | 2 (3.3%) | 10 (10.4%) | 0.225 |
| N-stage | N1 | 24 (40.0%) | 40 (41.7%) | |
| | N2 | 34 (56.7%) | 46 (47.9%) | |
| Clinical | M0 | 58 (96.7%) | 94 (97.9%) | 0.639 |
| M-stage | M1 | 2 (3.3%) | 2 (2.1%) | |
| Pathological | Tis | 1 (1.7%) | 2(2.0%) | 0.908 |
| T-stage | T0 | 9 (15.0%) | 16 (16.7%) | |
| | T1 | 3 (5.0%) | 4 (4.2%) | |
| | T2 | 17 (28.3%) | 25 (26.0%) | |
| | T3 | 30 (50.0%) | 46 (47.9%) | |
| | T4 | 0(0.0%) | 3 (3.1%) | |
| Pathological | N0 | 41 (68.3%) | 66 (68.8%) | 0.762 |
| N-stage | N1 | 10 (16.7%) | 22 (22.9%) | |
| | N2 | 5 (8.3%) | 8 (8.3%) | |
| Pathological | M0 | 59 (98.3%) | 95 (99.0%) | 1.000 |
| M-stage | M1 | 1 (1.7%) | 1 (1.0%) | |

Abbreviations: Pathological; T, tumor; N, node; M, metastasis

(2) Differential Gene Expression Analyses Between the Responder and the Non-Responder Groups

In the training cohort, differential gene expression analysis between responders (n=27) and non-responders (n=33) showed that 47/730 genes were differentially expressed with statistical significance (p<0.05). Of these 47 genes, 42 were selected after multivariate logistic regression (p<0.05). In responders, 28 genes were down-regulated and 14 genes were up-regulated (Table 2).

TABLE 2
Differentially expressed genes between responders
and non-responders in the training cohort

| Sr. No. | Gene | p-value | Fold-change |
|---|---|---|---|
| 1 | ID1 | 2.17E−02 | −2.78 |
| 2 | WNT11 | 1.99E−02 | −2.32 |
| 3 | NKD1 | 1.90E−03 | −2.27 |
| 4 | SFN | 2.45E−03 | −2.24 |
| 5 | LAMA5 | 8.51E−03 | −1.96 |
| 6 | CACNA1D | 5.06E−04 | −1.96 |
| 7 | IKBKB | 9.91E−03 | −1.88 |
| 8 | PLA2G4F | 5.89E−03 | −1.83 |
| 9 | CDK6 | 3.63E−03 | −1.79 |
| 10 | CIC | 1.21E−02 | −1.76 |
| 11 | SMARCB1 | 5.80E−04 | −1.75 |
| 12 | SRSF2 | 3.07E−03 | −1.73 |
| 13 | IKBKG | 1.51E−02 | −1.68 |
| 14 | BAP1 | 9.01E−03 | −1.68 |
| 15 | SPRY2 | 1.52E−03 | −1.65 |
| 16 | AXIN2 | 5.01E−03 | −1.65 |
| 17 | GNA11 | 6.73E−03 | −1.64 |
| 18 | FGFR4 | 1.69E−03 | −1.64 |
| 19 | FGFR3 | 1.05E−03 | −1.62 |
| 20 | HDAC10 | 1.08E−03 | −1.62 |
| 21 | SGK2 | 5.64E−03 | −1.6 |
| 22 | H3F3A | 2.42E−03 | −1.58 |
| 23 | EPHA2 | 1.74E−02 | −1.57 |
| 24 | U2AF1 | 4.56E−03 | −1.56 |
| 25 | ITGB4 | 3.14E−03 | −1.56 |
| 26 | TRAF7 | 6.07E−03 | −1.54 |
| 27 | CBL | 5.56E−03 | −1.53 |
| 28 | MAP2K2 | 1.99E−03 | −1.51 |
| 29 | BMP2 | 3.06E−02 | 1.5 |
| 30 | CDC14B | 5.16E−03 | 1.51 |
| 31 | CACNA2D1 | 1.13E−02 | 1.52 |
| 32 | ETV7 | 1.37E−02 | 1.55 |
| 33 | STAT1 | 1.89E−02 | 1.56 |
| 34 | ITGB3 | 2.98E−02 | 1.57 |
| 35 | FAS | 2.59E−02 | 1.58 |
| 36 | IL1R1 | 2.31E−02 | 1.61 |
| 37 | RASGRP1 | 3.42E−02 | 1.69 |
| 38 | IRS1 | 1.17E−02 | 1.7 |
| 39 | BMP8A | 1.77E−02 | 1.75 |
| 40 | IL12A | 3.73E−02 | 1.99 |
| 41 | CD40 | 1.51E−03 | 2.18 |
| 42 | IL2RB | 1.27E−02 | 2.33 |

Fold-change was calculated by dividing the mean expression level of responders by that of non-responders.

(3) Gene Signature Selection and Logistic Regression Analyses

A nine-gene signature was determined by k-fold cross validation of the 42 DEGs to identify the optimal gene combination. The nine genes were FGFR3, GNA11, H3F3A, IL12A, IL1R1, IL2RB, NKD1, SGK2, and SPRY2. As a result of 2-fold cross validation among all candidate gene signatures, the nine-gene signature consistently showed accuracy (86.9%) for differentiating between responders and non-responders (sensitivity=81.5% and specificity=84.8% in the training cohort) (Table 3).

TABLE 3
Gene signature candidates

| | | | Cross | | | |
|---|---|---|---|---|---|---|
| | | Logistic | Validation | | | |
| Sr. | | Regression | Accuracy | | Sensitivity | Specificity |
| No. | Gene Signature | p-value | (%) | AUC* | (%) | (%) |
| 1 | ETV7_H3F3A_HDAC10_1D1_IL1R1_SPRY2 | 7.60E−04 | 72 | 0.841 | 77.8 | 81.8 |
| 2 | H3F3A_IL12A_IL1R1_NKD1_PLA2G4F_SGK2 | 1.13E−04 | 79.7 | 0.868 | 77.8 | 87.9 |
| 3 | H3F3A_HDAC10_ID1_IL1R1_NKD1_SPRY2_STAT1 | 1.81E−04 | 75.7 | 0.878 | 77.8 | 93.9 |
| 4 | H3F3A_HDAC10_IL1R1_PLA2G4F_SGK2_TRAF7_WNT11 | 7.47E−04 | 57 | 0.822 | 81.5 | 75.8 |
| 5 | H3F3A_IL12A_IL1R1_NKD1_PLA2G4F_RASGRP1_SGK2 | 1.99E−04 | 77.3 | 0.871 | 81.5 | 90.9 |
| 6 | FGFR3_H3F3A_HDAC10_ID1_IL1R1_IL2RB_TRAF7_WNT11 | 4.65E−04 | 81 | 0.851 | 77.8 | 84.8 |
| 7 | FGFR3_GNA11_H3F3A_IL12A_IL1R1_IL2RB_NKD1_SGK2_SPRY2 | 2.56E−04 | 83.3 | 0.869 | 81.5 | 84.8 |
| 8 | FGFR3_H3F3A_IL12A_IL1R1_IL2RB_NKD1_SGK2_SPRY2_TRAF7 | 3.56E−04 | 73.7 | 0.870 | 77.8 | 84.8 |

*AUC: area under the curve

The association of the selected gene signature with clinicopathological features was investigated. Univariate analysis showed that gender and the selected gene signature were significantly and positively correlated with PCRT response. Pathological T-stage was significantly and negatively correlated with PCRT response (Table 4). Multivariate analysis was performed to assess the association between the gene signature, gender, and pathological T-staging. The results confirmed that the gene signature, gender, and pathological T-staging were independent predictors of PCRT response (Table 5).

TABLE 4
Univariate logistic regression analysis of the predictive value of the selected gene signature in PCRT responders (p < 0.05)

| | | | | | |
|---|---|---|---|---|---|
| Variable ¹ | N² | Coef³ | SE⁴(Coef) | Z-score | p-value |

Candidate genes

| | | | | | |
|---|---|---|---|---|---|
| FGFR3_GNA11_H3F3A_IL12A_IL1R1_IL2RB_NKD1_SGK2_SPRY2 | 60 | 3.204371 | 0.693663 | 4.619491 | 3.85E−06 |
| (low vs. high) | | | | | |

Clinicopathological features

| | | | | | |
|---|---|---|---|---|---|
| GENDER (Male vs. Female) | 60 | 1.170379 | 0.549265 | 2.130811 | 3.31E−02 |
| GRADED_DESCRIPTION (Moderate vs.Well) | 59 | −0.787079 | 0.783116 | −1.00506 | 3.15E−01 |
| CLIN_T_TNM (T2 vs. T3-T4) | 60 | −1.386294 | 1.185853 | −1.16903 | 2.42E−01 |
| CLIN_N_TNM (N0-N1 vs. N2) | 60 | −0.633724 | 0.528492 | −1.19912 | 2.30E−01 |
| CLIN_M_TNM (M0 vs. M1) | 60 | 16.8437 | 1696.73436 | 0.009927 | 9.92E−01 |
| PATH_T_TNM (Tis-T0-T1-T2 vs. T3) | 60 | −2.233592 | 0.605857 | −3.68666 | 2.27E−04 |
| PATH_N_TNM (N0-N1 vs. N2) | 56 | −0.04879 | 0.956183 | −0.05103 | 9.59E−01 |
| PATH_M_TNM (M0 vs. M1) | 60 | −15.39617 | 1455.39756 | −0.01058 | 9.92E−01 |

¹ Abbreviations: CLIN, clinical; PATH, pathological; T, tumor; N, node; M, metastasis.
²Coef, coefficient.
³SE, standard error.
⁴N, number of samples.

TABLE 5
Multivariate analysis of the association between the gene
signatures and gender and pathological tumor staging

| Variable ¹ | Odds ratio | 95% CI² | p-value |
|---|---|---|---|
| FGFR3_GNA11_H3F3A_IL12A_IL1R1_IL2RB_NKD1_SGK2_SPRY2 | 25.6 | 4.71-139.23 | 0.0002 |
| (low vs. high) | | | |
| GENDER (Male vs. Female) | 2.26 | 0.48-10.72 | 0.3048 |
| PATH_T_TNM (Tis-T0-T1-T2 vs. T3) | 0.08 | 0.02-0.46 | 0.0042 |

¹ Abbreviations: CLIN, clinical; PATH, pathological; T, tumor; N, node; M, metastasis.
² CI, confidence interval.

(5) Selected Gene Signature Validation

The selected gene signature distinguished PCRT responders from non-responders with an accuracy of 81.0% in our validation cohort (n=96) (Table 6).

TABLE 6
Evaluation of the clinical performance of the nine-gene signature to predict PCRT response in patients

| Gene signature | | FGFR3_GNA11_H3F3A_IL12A_IL1R1_IL2RB_NKD1_SGK2_SPRY2 |
|---|---|---|
| Logistic regression p-value | | 4.62 × 10⁻⁴ |
| Cross validation accuracy (%) | | 83.3 |
| Number of genes | | 9 |
| Training set | Accuracy (%) | 86.9 |
| | Sensitivity (%) | 81.5 |
| | Specificity (%) | 84.8 |
| | Response (%) | 81.5 |
| Validation set | Accuracy (%) | 81.0 |
| | Sensitivity (%) | 82.3 |
| | Specificity (%) | 79.4 |
| | PPV¹ (%) | 87.9 |
| | NPV² (%) | 71.1 |

¹PPV, positive predictive value.
²NPV, negative predictive value.

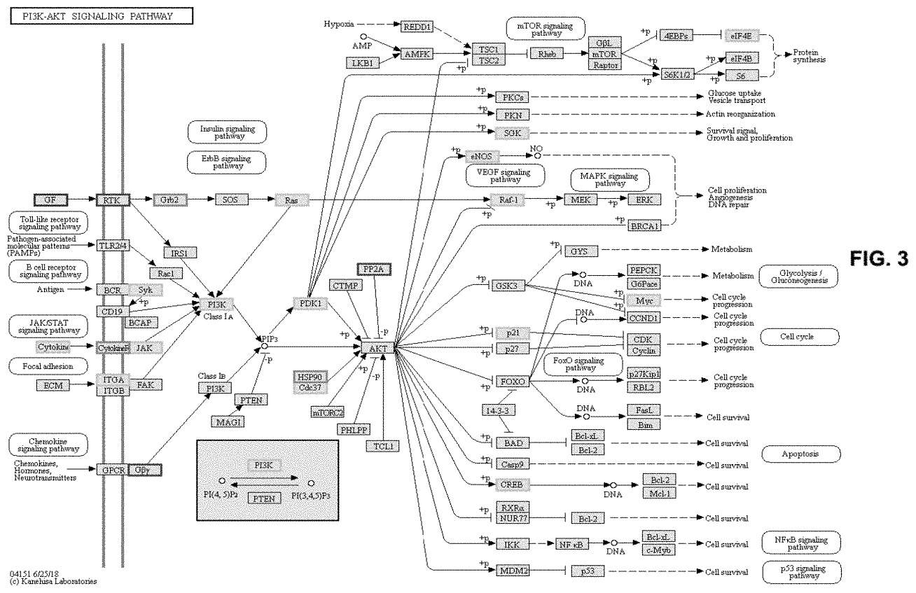

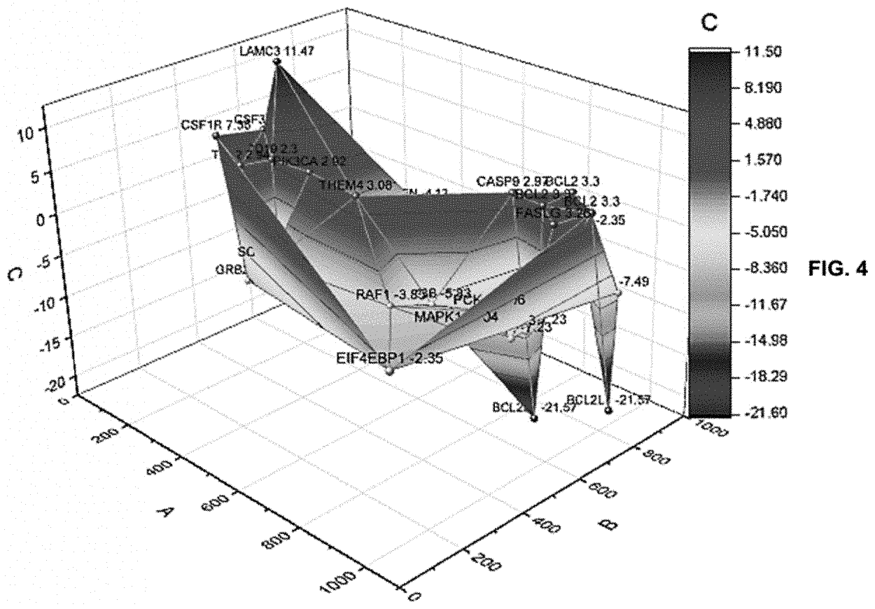

The nine-gene signature predictive of PCRT response was highly related to KEGG signal transduction pathways including cancer-related pathways, PI3K-Akt signaling pathways (FIG. 3 and FIG. 4), proteoglycans in cancer, human cytomegalovirus infection, and human papillomavirus infection. High interaction frequency genes related to the gene signature included GRB2, HSP90AA1, and HSP90AB1 (Table 7).

TABLE 7
Gene signature-related pathways and high interaction
frequency genes associated with PCRT responders

| | Name of pathways/High |
|---|---|
| | interaction frequency genes |
| Pathways | Pathways in cancer |
| | PI3K-Akt signaling pathway |
| | Proteoglycans in cancer |
| | Human cytomegalovirus infection |
| | Human papillomavirus infection |
| High interaction | GRB2 |
| frequency genes | HSP90AA1 |
| | HSP90AB1 |

(4) Calculation of Treatment Benefit Prediction Score (TBPS) Through Logistic Regression Analysis

The regression coefficient values for each gene obtained through the univariate logistic regression analysis on the combination of the nine genes selected by the above method (i.e., FGFR3, GNA11, H3F3A, IL12A, IL1R1, IL2RB, NKD1, SGK2, and SPRY2) are shown in the following Table 8.

TABLE 8

| Gene | Regression coefficient value |
|---|---|
| FGFR3 | −0.006697 |
| GNA11 | −0.001805 |
| H3F3A | −0.000373 |
| IL12A | 0.063996 |
| IL1R1 | 0.015269 |
| IL2RB | 0.017445 |
| NKD1 | −0.003099 |
| SGK2 | −0.004739 |
| SPRY2 | −0.002763 |

A Treatment Benefit Prediction Score (TBPS) was calculated according to the following equation, using the normalized expression levels of the respective 9 genes (i.e., FGFR3, GNA11, H3F3A, IL12A, IL1R1, IL2RB, NKD1, SGK2, and SPRY2) obtained with nCounter (Nanostring Technologies, Seattle, Wash.) and the regression coefficient values for each gene.

TBPS=C_{FGFR3}*G_{FGFR3}+C_{GNA11}*G_{GNA11}+C_{H3F3A}*G_{H3F3A}+C_{IL12A}*G_{IL12A}+C_{IL1R1}*G_{IL1R1}+C_{IL2RB}*G_{IL2RB}+C_{NKD1}*G_{NKD1}+C_{SGK2}*G_{SGK2}+C_{SPRY2}*G_{SPRY2}

In the above equation, C_{gene} represents the regression coefficient value of the corresponding gene; and G_{gene} represents the normalized expression level of the corresponding gene which was obtained with nCounter (Nanostring Technologies, Seattle, Wash.). Thus, from the results of Table 8, the TBPS can be also calculated according to the following equation.

TBPS=(−0.006697)*G_{FGFR3}+(−0.001805)*G_{GNA11}+(−0.000373)*G_{H3F3A}+(0.063996)*G_{IL12A}+(0.015269)*G_{IL1R1}+(0.017445)*G_{IL2RB}+(−0.003099)*G_{NKD1}+(−0.004739)*G_{SGK2}+(−0.002763)*G_{SPRY2}

The calculated TBPS value as described above is −7.269813, which can be used as a threshold capable of predicting a response to PCRT. That is, after measuring the expression levels of FGFR3, GNA11, H3F3A, IL12A, IL1R1, IL2RB, NKD1, SGK2, and SPRY2 genes in a rectal cancer patient; when the TBPS value calculated according to the above equation is greater than −7.269813, the patient can be classified to a patient who exhibits a response to PCRT (i.e., a patient who exhibits susceptibility to PCRT) and when the TBPS value is −7.269813 or less, the patient can be classified to a patient who does not exhibit a response to PCRT (i.e., a patient who does not exhibit susceptibility to PCRT).

3. Discussion

In this study, we identified and validated a nine-gene signature capable of predicting the response to PCRT in LARC patients. This nine-gene signature has three main advantages over previously reported predictive signatures: 1) it shows a better accuracy for predicting the response to PCRT than previously reported signatures; 2) gene expression analysis can be performed using FFPE samples and FDA-approved hardware and reagents; and 3) the nine-gene signature was validated in larger cohorts than those used in previous studies. Therefore, the method is readily applicable to the clinical setting.

The nine-gene signature capable of predicting the response to PCRT with high accuracy has two important clinical implications. First, good responders identified using the nine-gene signature can be treated with PCRT, which may lead to rectal-sparing surgery. Local excision or deferral of surgery is sometimes used to avoid surgical complications associated with radical resection and to reduce the risk of stoma formation, which may compromise quality of life. Second, the identification of poor responders would be beneficial for the treatment of patients with LARC because it would prevent exposure to toxic and inefficient radiation therapy in this population. In addition, the delay of surgical treatment because of ineffective PCRT could be avoided. This tailored approach based on the molecular characteristics of LARC could improve the overall survival and quality of life of patients with LARC.

The nine genes included in the signature are dysregulated in cancer (Waaler J, is et al. Novel synthetic antagonists of canonical Wnt signaling inhibit colorectal cancer cell growth. Cancer Res. 2011; 71: 197-205; Marshall K W, et al. A blood-based biomarker panel for stratifying current risk for colorectal cancer. Int. J. Cancer 2010; 126: 1177-86; Chang Y T, et al. Verification of gene expression profiles for colorectal cancer using 12 internet public microarray datasets. World J. Gastroenterol. 2014; 20: 17476-82; Stanilov N S, et al. Monocytes expression of IL-12 related and IL-10 genes in association with development of colorectal cancer. Mol. Biol. Rep. 2012; 39: 10895-902; Fromme J E, et al. FGFR3 mRNA overexpression defines a subset of oligometastatic colorectal cancers with worse prognosis. Oncotarget 2018; 9: 32204-32218; Feng Y H, et al. MicroRNA-21-mediated regulation of Sprouty2 protein expression enhances the cytotoxic effect of 5-fluorouracil and metformin in colon cancer cells. Int. J. Mol. Med. 2012; 29: 920-6; Ayoubi H A, Investigation of the human H3.3B (H3F3B) gene expression as a novel marker in patients with colorectal cancer. J. Gastrointest. Oncol. 2017; 8:64-69; Feng Y H, et al. Deregulated expression of sprouty2 and microRNA-21 in human colon cancer: Correlation with the clinical stage of the disease. Cancer Biol. Ther. 2011; 11: 111-21). FGFR3 is frequently overexpressed in rectal cancer patients and functions as an oncogene by promoting cell proliferation and migration (Fromme J E, et al. FGFR3 mRNA overexpression defines a subset of oligometastatic colorectal cancers with worse prognosis. Oncotarget 2018; 9: 32204-32218). GNA11 encodes a G protein alpha subunit and is involved in carcinogenesis in various cancers (Shoushtari A N, et al. GNAQ and GNA11 mutations in uveal melanoma. Melanoma Res. 2014; 24: 525-34; Van Raamsdonk C D, et al. Mutations in GNA11 in uveal melanoma. N. Engl. J. Med. 2010; 363: 2191-9). SPRY2 overexpression contributes to rectal cancer development by promoting EMT (Zhang Q, et al. Atypical role of sprouty in colorectal cancer: sprouty repression inhibits epithelial-mesenchymal transition. Oncogene 2016; 35: 3151-62). SGK1 is involved in several physiological processes such as migration and proliferation and is upregulated in colorectal cancer (Eide P W, et al. NEDD4 is overexpressed in colorectal cancer and promotes colonic cell growth independently of the PI3K/PTEN/AKT pathway. Cell. Signal. 2013; 25: 12-8). Therefore, the nine-gene signature is not only a practical tool for predicting the response to PCRT, but also a mechanistic link to the underlying biology of LARC.

In summary, we identified a nine-gene signature capable of predicting the response to PCRT in patients with LARC. This gene signature is readily applicable to the clinical setting using FFPE samples and FDA-approved hardware and reagents. Tailored treatment approaches in good and poor responders to PCRT may improve the oncologic outcomes of patients with LARC.

1. An analytical method of providing an information for diagnosis of a patient who exhibits a response to preoperative chemoradiotherapy in a rectal cancer patient, the method of which comprises measuring expression levels of FGFR3, GNA11, H3F3A, IL12A, IL1R1, IL2RB, NKD1, SGK2, and SPRY2 genes, in carcinoma tissue samples which are externally discharged from the rectal cancer patient, respectively.

2. The analytical method according to claim 1, wherein the rectal cancer patient is a locally advanced rectal cancer patient.

3. The analytical method according to claim 1 or 2, wherein the carcinoma tissue samples which are externally discharged from the rectal cancer patient are formalin-fixed paraffin-embedded carcinoma tissue-derived biopsy samples.
